(19) United States
(12) Patent Application Publication
(10) Pub. No.: US 2017/0150970 A1
(43) Pub. Date: Jun. 1, 2017

(54) Device for use in performing organ occlusions, especially an intrauterine tracheal occlusion in the treatment of a congenital fetal diaphragmatic hernia

(71) Applicant: Universitätsklinikum Halle (Saale), Halle (Saale) (DE)

(72) Inventor: Michael Tchirikov, Halle (Saale) (DE)

(21) Appl. No.: 15/365,437

(22) Filed: Nov. 30, 2016

(30) Foreign Application Priority Data
Nov. 30, 2015 (EP) 15196990.4

(51) Int. Cl. A61B 17/12

(52) U.S. Cl. CPC A61B 17/12104; A61B 17/1204; A61B 17/12136; A61B 2017/1205; A61B 2017/00557

(57) ABSTRACT

The present invention concerns a device for use during organ occlusions in humans or mammals, especially for the performing of an intrauterine tracheal occlusion in the course of the treatment of a congenital fetal diaphragmatic hernia, comprising a balloon body (1) which can be filled with a fluid with a balloon shell (2), forming a lumen to hold the fluid, and a balloon mouth (3) for introducing the fluid into the lumen, a separate auxiliary catheter for the placement of the balloon body (1) in the organ segment being treated and/or for the inflation of the balloon body (1) with the fluid, wherein the balloon body (1) is connected to a flexible, elongated prolongation element (4).

The present invention concerns a device for use during organ occlusions in humans or mammals, especially for the performance of an intrauterine tracheal occlusion during treatment of a congenital fetal diaphragmatic hernia.

Artificially induced occlusions of organs in humans or mammals are the preferred method of cure for the prevention or treatment of a number of diseases, which presents special challenges to the development of medical products. This applies in particular to fetal occlusions, as there are substantial treatment risk in this case, which need to be weighed carefully against the customary benefits. The congenital diaphragmatic hernia is a rare and serious malformation with a frequency of 1 or 2 out of 5000 pregnancies, leading prenatally to a malformation of the fetal lungs, which leads for example to a displacement of the abdominal organs, especially the liver, in the thoracic cavity (Skari H, et al., Congenital diaphragmatic hernia: a meta-analysis of mortality factors. J Pediatr Surg 2000; 35:1187-1197; Rotter et al., Fetal lung and diaphragm development in congenital diaphragmatic hernia. Semin Perinatol 2005; 195:1720-1728). On the other hand, postnatal defects are often easily surgically treatable.

The affected lungs are distinctly hypoplastic and exhibit a reduced surface for gas exchange as well as a low number of alveoli, which moreover have thickened walls (Areechon W et al.: Hypoplasia of lung with congenital diaphragmatic hernia; Br Med J 1963:5325:230-233). This, due to the resulting pulmonary hypoplasia and pulmonary hypertonia, often leads to a severe postnatal respiratory failure, which despite the increasing availability of an extracorporeal membrane oxidation (ECMO) results in the death of the affected children in around half of the cases (Beresford M W et al: Outcome of congenital diaphragmatic hernia. Pediatr Pulmonol 2000; 30: 249-256). The children who survive often have a bronchiopulmonary dysplasia.

Prenatal diagnostics for diaphragmatic hernia involves for example a measurement of lung size by means of ultrasound or magnetic resonance tomography and a determination of the position of the abdominal organs, such as the liver position. Depending on the anticipated postnatal course, one must therefore consider whether to perform an abortion of the pregnancy or a prenatal treatment, given the quite high mortality and morbidity. The goal of such a prenatal intervention is not a correction of the anatomical defect occasioned by the displacement of the abdominal organs, but rather a minimization of the pulmonary malformation as well as the pulmonary hypoplasia. This is made possible by fetoscopic endoluminal tracheal occlusion (Deprest J et al: FETO Task Group: Fetoscopic tracheal occlusion (FETO) for severe congenital diaphragmatic hernia: evolution of a technique and preliminary results. Ultrasound Obstet Gynecol 2004; 24: 121-126). A temporary tracheal occlusion constitutes a congenital obstruction of the respiratory pathway during which an increased lung growth occurs. This is because a “closure” of the trachea does not allow any drainage of the fluid produced in the lungs. This causes an increased distension of the lungs, which in turn leads to a heightened expression of various growth factors and thus an intensified lung growth (DiFiore J W et al: Experimental fetal tracheal ligation reverses the structural and physiological effects of pulmonary hypoplasia in congenital diaphragmatic hernia. J Pediatr Surg 1994; 29: 248-256; Deprest J A et al: Tracheoscopic endoluminal plugging using an inflatable device in the fetal lamb model. Eur J Obstet Gynecol Reprod Biol 1998; 81: 165-169).

During the fetoscopic therapy, in the context of an invasive procedure a fetoscope is introduced via the mother's abdominal wall into the uterus and there via the mouth of the child into the trachea (Deprest J A et al: Fetal surgery for congenital diaphragmatic hernia is back from nevergone. Fetal Diagn Ther 2011; 29: 6-17). After this, a balloon is shoved in to just before the carina, deployed and put in place with liquid. The fetoscopic canal of the trochar can be used for this under 3-D ultrasound control (Tchirikov M. Successful tracheal occlusion using ultra-thin fetoscopic equipment combined with real-time 3D ultrasound. Eur Surg Res 2009; 43(2):204-7).

The balloon prevents a drainage of lung fluid and is generally left in the child's trachea for a period of 4 to 6 weeks, after which it is removed in the same way. Thus far, it has been possible to increase the survival rate with this treatment method from the expected 10-20% to around 50%, and a substantial reduction in postnatal morbidity has been achieved.

However, the high mortality and morbidity rates continue to deter many mothers from the use of a fetoscopic endoluminal tracheal occlusion (FETO), so that they prefer to end the pregnancy, because the classical treatment of a congenital diaphragmatic hernia by a tracheal occlusion with a balloon is risky, due to several causes. Thus, a second fetal surgical procedure is necessary to remove the intratracheal balloon, generally occurring in week 34 and 35 of pregnancy (WP). Moreover, there are the risks of an iatrogenic amniorrhexis as well as a premature birth. The iatrogenic amniorrhexis represents a frequent complication, which is also definitively attributable to the devices and balloons used and the accompanying traumatization of the amnial membrane. A further problem involves the much more difficult safeguarding of the child's respiratory passages with the balloon in situ in the case of an unplanned delivery prior to the second procedure (Harrison M R, Keller R L, Hawgood S S et al. A randomised trial of fetal endoscopic tracheal occlusion for severe fetal congenital diaphragmatic hernia. N Engl J Med 2003; 349:1916-1924; Deprest J, Jani J, Gratacos E et al. Fetal Intervention for congenital diaphragmatic hernia: the European experience. Semin Perinatol 2005; 29:94-103). In this case, an EXIT (“ex utero intrapartum treatment”) procedure constitutes the modus operandi, during which the head of the child is first delivered by a uterotomy during a Caesarian section and then a safeguarding of the respiratory passages is done intrapartally by removal of the balloon and intubation of the child, and only then is the child's body delivered. However, this procedure involves substantial risks, which is often attributable to a difficult removal of the tracheal balloon and difficult intubation conditions. If the balloon is not removed at once from the trachea after the birth, the newborn will die.

One is confronted with similar problems of balloon removal or even total absence of non-medication treatment methods for other indications, such as nosebleeds or intracorporeal organ bleeding. Thus, with certain diseases such as tumor diseases, considerable organ-specific bleeding occurs, which can only be staunched in the hospital at considerable expense. By introducing a balloon body into the affected organ cavity (for example, into the bleeding nose or into the uterus), such organ bleeding can be drastically reduced, for example, by the balloon body exerting pressure on the affected blood vessels of the portion of the organ, thereby alleviating or even entirely preventing the bleeding.

Although there are many known balloon implants, the removal of the balloon at the end of the treatment segment for the indications involving organ occlusions constitutes a substantial problem for the surgeon and/or for the patient. Thus, for example, DE 103 02 241 A1 describes a balloon implant for occluding of aneurisms. The balloon is made from a textile fabric or knitted fabric and contains a procoagulant liquid, such as fibrin or a synthetic tissue adhesive. EP 0 876 166 B1 describes a biomaterial composition for the filling and covering of cavities or lumens of a body, during which an emplacement of the implants is done under control. In addition, there are a number of balloon catheters, such as are described in DE 197 32 965 A1 for fixation in hollow organs, but these medical products are not suitable for organ occlusions, especially for a difficult procedure in the context of an intrauterine tracheal occlusion during the treatment of a congenital diaphragmatic hernia.

Against this background, the problem which the present invention proposes to solve is to provide a medical product for performing an organ occlusion, especially a balloon suitable for an intrauterine tracheal occlusion, which is easier to locate in the course of a fetoscopic endoluminal procedure or which is easily removable by the surgeon or the patient themselves.

This problem is solved by a device with the features of claim 1. Preferred embodiments are reflected in the subclaims.

The device according to the invention is designed for use during organ occlusions in humans or mammals, especially for the performing of an intrauterine tracheal occlusion in the course of the treatment of a congenital fetal diaphragmatic hernia. The device comprises a balloon body which can be filled with a fluid, consisting of a balloon shell, forming on its inside a lumen to hold the fluid, as well as a balloon mouth. The balloon body according to the invention is connected to a flexible, elongated prolongation element. Preferably, the fluid for an intrauterine application is a physiological solution, such as a NaCl solution. Depending on the application and the kind of organ occlusion, however, a filling with a gas or a gas mixture such as air is also possible. The filling is done through the balloon mouth, which is formed either by the balloon shell itself or—in a variant embodiment—by the proximal end of the prolongation element.

The device moreover comprises a separate auxiliary catheter for the placement of the balloon body in the organ section being treated and/or for the inflation of the balloon body with the fluid. In particular during the fetal surgery removal and in event of an unplanned EXIT procedure, the removal of the balloon represents a serious problem, which also contributes not least of all to the still high morbidity rates of the fetus or the newborn. According to the invention, therefore, it is provided that the balloon body is connected to a flexible elongated prolongation element, which extends as a filament, in the manner of a tail from the balloon body, i.e., the balloon shell or the balloon mouth. The flexible elongated prolongation element connected to the balloon body is longer in its lengthwise dimension by a multiple of the length of the balloon body, i.e., 2-, 3-, 4-, 5-, 6-, 7-, 8-, 9-, 10-times or more.

By a “flexible prolongation element” in the sense of the invention is meant an elongated body with a length to width ratio of at least 4 to 1, preferably at least 10 to 1 or more. Flexible means that the prolongation element when necessary can be wound up manually, guided freely (unlike a wire, for example), and is not rigid. Preferably the flexible elongated prolongation element consists of solid material or is internally hollow for at least a section. Preferably it involves a filament, a thread, a fiber, a fiber blend, a cord, a hose, a yarn or a ribbon. In one variant using solid material, the prolongation element is not in fluid communication with the lumen of the balloon body. Alternatively, the prolongation element can comprise a channel for at least one section, which serves for example to guide the auxiliary catheter for the filling of the balloon. But the channel can also extend for the full length of the prolongation element.

In another preferred variant, the flexible elongated prolongation element is joined by force locking to the balloon mouth or to the balloon shell. Thus, the prolongation element can be led in the manner of a thread as an extension from the balloon mouth or the balloon shell. In another embodiment, the flexible prolongation element can close the balloon mouth, for example, in that a thread is wound around the balloon mouth, or in that a separate closure device for the balloon mouth is provided in a prolongation element which is hollow for at least one section.

The balloon mouth itself can also be formed by the balloon shell or by a piece of tubing inserted in the balloon body. Preferably a valve flap is integrated in the balloon mouth. The valve flap is, for example, a plate or a fluid-tight membrane which closes the channel in fluid-tight manner.

When the prolongation element comprises a channel which stands in fluidic communication with the lumen of the balloon body, then the valve flap is integrated in the channel and it can open or close the channel upon introducing and removing the auxiliary catheter. The valve flap is preferably situated at the distal end of the channel running in the prolongation element, i.e., at the end where the auxiliary catheter is introduced.

Insofar as the prolongation element is fashioned for its entire length as a hose with a channel or cavity extending therein, the valve flap can also be located at the distal end of the prolongation element. In this variant, it would be sufficient for the flap to close the channel fluid-tight after the filling of the balloon. A deflation of the balloon is possible when the distal end of the prolongation element is cut off behind the valve flap (i.e., at the balloon side), which leads to an immediate escaping of fluid from the balloon body and significantly simplifies the surgical procedure. Preferably, the valve flap is made from the same material as the prolongation element.

In one alternative variant it can be provided that the balloon mouth or another part of the balloon system opens when the prolongation element is pulled with a given force >0.5 N, preferably a force between 0.5 and 10 N. This has a significant benefit in the case of an intrauterine tracheal occlusion: thus, it is known that the fetus develops a pronounced playing behavior already in the uterus, so that it might itself remove the tracheal occlusion at the end of the pregnancy by opening the balloon mouth via the threadlike flexible prolongation element. The balloon would then be deflated by the escape of the fluid. Thus, an opening is possible only when the fetus has developed the required strength for the opening of the balloon. Moreover, in this way an unintentional opening of the valve and thus a deflation of the balloon can be prevented.

In an intrauterine tracheal occlusion the balloon is inserted into the trachea of the fetus during a particular stage of development. As the pregnancy proceeds, the trachea expands in the course of the child's development, so that the removal of the balloon becomes easier. The emptying of the balloon can be done either by opening the valve flap or by ripping open the balloon mouth or the balloon body via the prolongation element.

The balloon according to the invention affords the possibility of an easier removal by the surgeon or by the patient themselves, depending on the particular organ occlusion. It is preferably provided that the balloon mouth or the balloon body opens itself for the removal. For this, in one preferred variant, a predetermined breaking point is provided in the balloon mouth or on the balloon body, which breaks open when a pulling force is exerted on the prolongation element. Thus, it can be provided that the balloon shell or the balloon mouth will rip open at the predetermined breaking point upon applying a force of >0.5 N.

In an alternative variant embodiment, the prolongation element according to the invention comprises a closure flap at the distal end. For the removal of the balloon, the flap is cut off to let the fluid or liquid drain from the balloon via the catheter. After this, the balloon is removed by being pulled out from the trachea.

In the course of a fetoscopic removal, the prolongation element offers substantial benefits as compared to a traditional balloon, since the prolongation element and its position can be localized and removed very easily. In this way, the operating time and the attendant fetal surgery risks are substantially reduced.

Depending on the indication and the type of organ occlusion being performed, the dimensions of the balloon body and the threadlike flexible prolongation element may vary. Preferably, the balloon body holds a volume between 0.25 and 9 ml, preferably between 1 and 3 ml. The opening diameter of the balloon mouth varies preferably between 1.0 and 6.0 mm, preferably between 1.5 and 3.0 mm. The balloon body comprises in its width (B) and its length dimension (L) preferably a ratio B:L between 7×10 mm and 70×120 mm, preferably between 7×10 and 15×30 mm.

The balloon is deployed with the fluid via the introduction of a separate auxiliary catheter and the fluid is held in the lumen of the balloon body by the fluid-tight closure flap. By a pulling on the prolongation element, the valve flap and thus the balloon mouth can be opened, so that the fluid can escape from the lumen of the balloon body. In one preferred embodiment, the prolongation element is connected directly to the valve flap in the balloon mouth by force locking. In an alternative variant embodiment, the valve flap can be activated by the surgeon or by the patient themselves via the prolongation element, for example, by applying a pulling force.

The balloon shell of the balloon body itself consists preferably of a polyurethane, a polyurethane-polyvinyl chloride blend or latex. Preferably, the threadlike flexible prolongation element is a polypropylene thread connected to the balloon mouth.

The present invention also concerns the use of the above-described device for the production of a medical product for use in organ occlusions in humans or mammals, preferably during an intrauterine tracheal occlusion for the prevention and treatment of a congenital fetal diaphragmatic hernia. As described, intrauterine fetal tracheal occlusion (FETO) represents a prenatal therapy option in the treatment of a congenital diaphragmatic hernia with poor prognosis, the most frequent complication being an iatrogenic amniorrhexis with the concomitant risks. Thanks to the use of a balloon with a threadlike flexible prolongation element fashioned thereon, the risk can be drastically reduced by a minimization of the amnial injury and possibly also by a reduction in the necessary procedures. The device according to the invention constitutes a substantial improvement in an intrauterine tracheal occlusion, since with a minimization of the therapy risks the indication for the operation can be broadened in regard to a prediction of the outcome and thus far more children can profit from the fetal surgery therapy. Thanks to the use of the balloon according to the invention in connection with a FETO, a distinctly better outcome is achieved as compared to a conservative management.

However, the device according to the invention is not only suitable for a fetal surgery therapy, such as that in the course of a tracheal occlusion for the treatment or prevention of a congenital fetal diaphragmatic hernia, but also for a number of other indications calling for an organ occlusion by means of a balloon body. This includes, for example, an occlusion of the upper respiratory passages, such as that in the course of a hemostasis occurring during nosebleeds or bleeding from the urogenital tract.

Such bleeding can be alleviated or prevented by the introducing of a deployed balloon into the affected organ segment, i.e., the balloon system according to the invention can also successfully replace a classical tamponade. However, such bleeding can also occur due to traumatic injuries, such as bruises or abrasions of the nasal mucous membrane or due to ulcers, varices, polyps or inflammation (e.g., rhinitis). In addition, general stress situations can also lead to bleeding, such as hemophilia, certain forms of anemia, high blood pressure, kidney and liver disease, leukemia, scurvy, infectious diseases, etc. The balloon body according to the invention, outfitted with a threadlike flexible prolongation element, is thus generally suitable as a medical product in the prevention or treatment of bleeding by organ occlusions, such as bleeding of the esophagus or the auditory canals.

The device according to the invention can be used in the course of palliative medicine for bleeding from the urogenital tract.

In addition, the device according to the invention can also be used for slow dilatation in the event of an organ constriction or scar formation, such as that of the esophagus, the urethra, the vagina, etc. Accordingly, the device according to the invention is also suitable for use in the course of a plastic surgery procedure or a plastic reconstruction.

Due to the high morbidity and the complication of an iatrogenic amniorrhexis associated with a fetoscopic positioning of a tracheal balloon, however, the use of the device according to the invention as a medical product in the course of an intrauterine fetal tracheal occlusion is a preferred area of application, since the morbidity rates of the affected patients here can be significantly lowered, since the finding and/or the removal of the tracheal balloon can be significantly improved as compared to the former procedure. The use of a threadlike flexible elongated prolongation element enables a fetoscopic removal, such as was not possible heretofore. The operation times and the attendant surgical procedures are significantly reduced, so that the morbidity risk and possible complications can also be significantly decreased. In one preferred variant, the balloon body or the balloon shell and also the prolongation element can consist of a biodegradable material, so that in the course of an intracorporeal balloon removal at first the balloon is deflated via the prolongation element and then a decomposition of the balloon material or the prolongation element occurs.

The invention will be explained more closely in the following sample embodiments, different variants of the balloon according to the invention being shown in the drawings.

### FIGURES

FIG. 1 shows a first sample embodiment of the device according to the invention (without auxiliary catheter), consisting of a balloon body 1, a balloon shell 2 and a balloon mouth 3. On the balloon mouth 3 there is located a threadlike flexible prolongation element 4 in the form of a polypropylene thread, which is wound around the balloon mouth 3. The prolongation element 4 in the variant depicted is not connected to the lumen of the balloon body and not hollow on the inside.

FIG. 2 shows another variant embodiment, in which a channel 6 runs for a section inside the flexible elongated prolongation element 4 at its proximal end, being in fluidic communication with the lumen of the balloon body 1. At the distal end of the channel 6 there is provided a closure device, in order to prevent an escape of fluid from the balloon. The opening 7 of the channel 6 at the distal end furthermore serves for introducing the auxiliary catheter for the positioning and/or filling of the balloon.

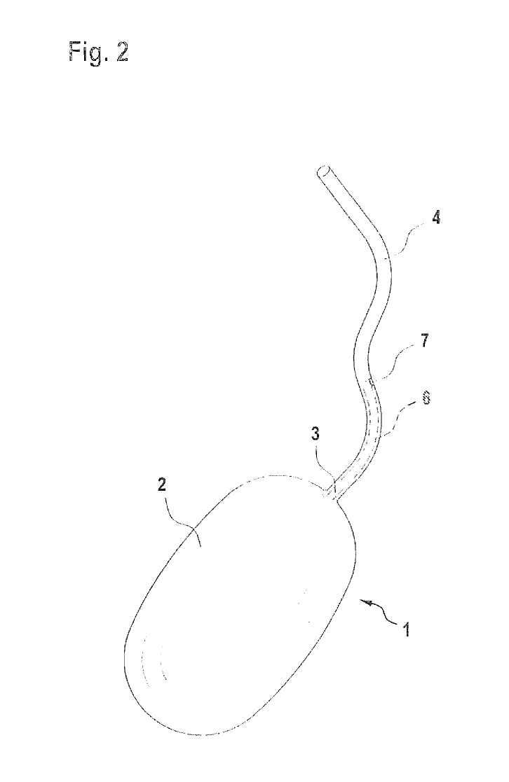

FIG. 3 shows another variant embodiment, in which a channel 6 runs in the prolongation element 4 for the entire length of the prolongation element 4. At the distal end of the prolongation element a valve flap 5 is arranged in the channel 6. The emptying of the balloon is done for example by cutting off the distal end of the prolongation element 4 behind the valve flap 5.

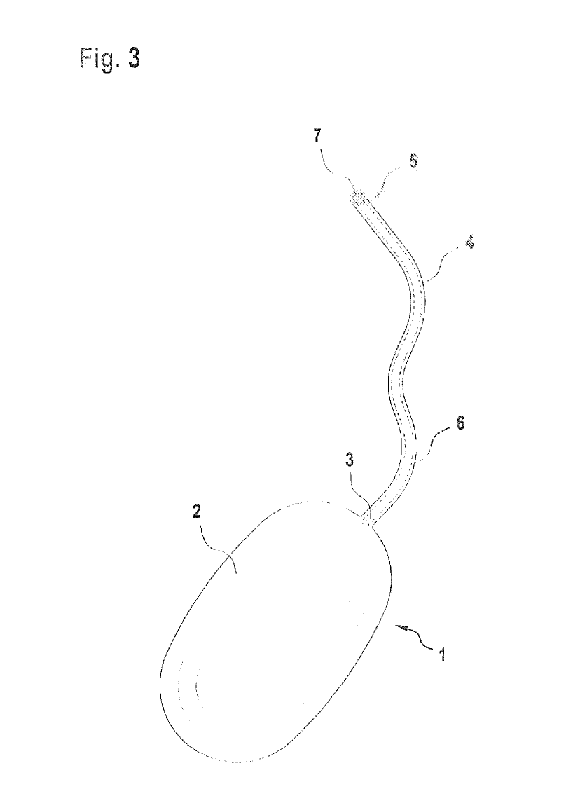

### CLINICAL TRIAL

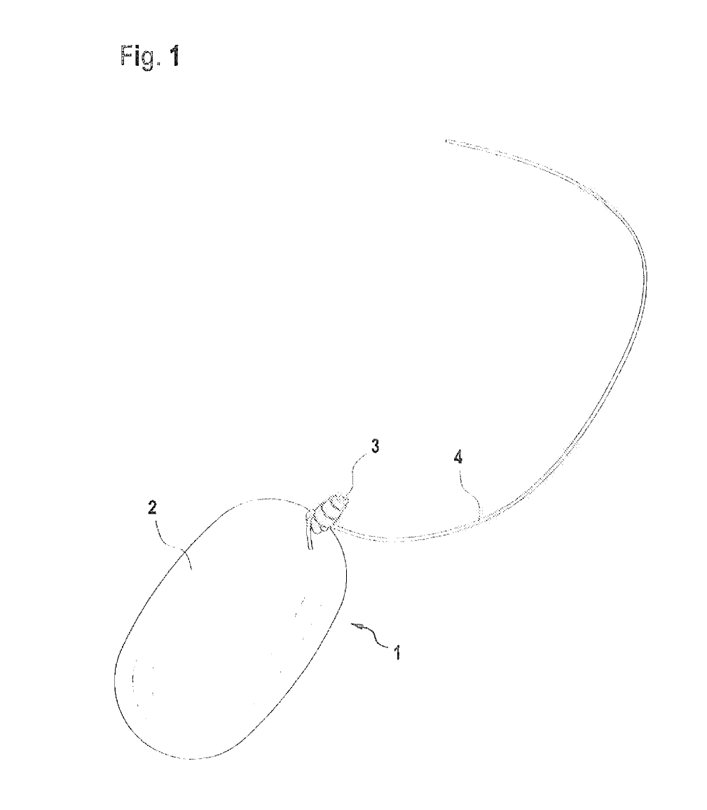

In the context of a trial, patients with a sonographically diagnosed, pronounced fetal diaphragmatic hernia with an o/e LV ratio (“observed/expected lung volume ratio”, MRT finding) of 15% were subjected to a treatment with the device according to the invention. A balloon per FIG. 1, outfitted with a polypropylene thread, was used to perform an intrauterine tracheal occlusion. The balloon body 1 consists of a latex balloon with a volume of 2.5 ml and a size of 12×28 mm. At the balloon mouth 3, a prolongation element 4 in the form of a polypropylene thread (monophil 2-0 polypropylene, 5/0) with a length of 7 cm was attached. Usually the closure of the fetal trachea was done intrauterine between week 26 and 28 of pregnancy (WP), whereupon the increased hydrostatic pressure in the bronchial system rises. The removal of the intratracheal balloon was done in the course of a second fetal surgery procedure between WP 34 and 35. After this, a fetal surgery treatment was performed after WP 28+4. The procedure was done with the aid of a 1.2 mm fetoscope under 3-D ultrasound control.

The fetus was intubated with no problems up to the point of the Bifurcatio trachae. The optics were temporarily removed and the balloon introduced into the fetal trachea with the aid of an auxiliary catheter under ultrasound control (Tchirikov M, Gatopoulos G, Strohner M, Puhl A, Steetskamp J. Two new approaches in intrauterine tracheal occlusion using an ultrathin fetoscope. Laryngoscope. 2010 Feb; 120(2):394-8). After the balloon with the threadlike flexible prolongation element 4 was put in place across the bifurcation, the deployment of the balloon body 1 was done with 2.5 ml of NaCl solution. During this process, the localization of the balloon outfitted with the prolongation element 4 and the condition of the fetus was monitored by sonography. Then the auxiliary catheter was removed and replaced with the optics. Sonographic imaging of the balloon in situ shows that the tracheal balloon with the prolongation element 4 was in the desired position.

In the further course, the observed/expected-LV ratio increased from 15% to 134%. Two months after the insertion, the removal of the tracheal balloon was done. In this process, it was easy to localize the polypropylene thread during the fetoscopic removal. The subsequent delivery was done per sectionem (“Caesarian”). The congenital diaphragmatic hernia was successfully operated upon the following day. After a period of one year, the child showed no permanent damage upon examination.

Thanks to the connection of the balloon body 1 to a threadlike flexible prolongation element 4, such as a polypropylene thread, the finding and the removal of the tracheal balloon can be greatly facilitated. In this way, the operation time was significantly reduced as compared to the classical method.

The connection of the balloon to a prolongation element 4 furthermore affords an intrauterine possibility of balloon removal by the fetus itself. In this variant embodiment, a second fetoscopic surgical procedure in WP 34 can be avoided entirely. The device according to the invention thus also enables an extending of the time of action of a tracheal occlusion on the growth of the lungs up to the time of delivery. Yet also with other indications calling for an organ occlusion, such as an occlusion of the upper respiratory passages for hemostasis or of other organs during organ bleeding, the patient can himself remove the balloon body 1 from the affected treatment area or the organ by pulling on the prolongation element 4. Thus, a removal of the balloon during these indications is much easier than was possible with traditional balloons.

1. Device for use during organ occlusions in humans or mammals, especially for the performing of an intrauterine tracheal occlusion in the course of the treatment of a congenital fetal diaphragmatic hernia, comprising
a balloon body (1) which can be filled with a fluid with a balloon shell (2), forming a lumen to hold the fluid, and a balloon mouth (3) for introducing the fluid into the lumen,
a separate auxiliary catheter for the placement of the balloon body (1) in the organ segment being treated and/or for the inflation of the balloon body (1) with the fluid, characterized in that the balloon body (1) is connected to a flexible, elongated prolongation element (4).

2. Device according to claim 1, characterized in that the flexible elongated prolongation element (4) involves a filament, a thread, a fiber, a fiber blend, a cord, a hose, a yarn or a ribbon.

3. Device according to claim 1, characterized in that the flexible elongated prolongation element (4) is connected by force locking to the balloon mouth (3) or to the balloon shell (2).

4. Device according to claim 3, characterized in that the flexible elongated prolongation element (4) closes the balloon mouth (3).

5. Device according to claim 4, characterized in that the flexible elongated prolongation element (4) opens up the balloon mouth (3) when a pulling force of >0.5 N is applied.

6. Device according to claim 1, characterized in that a valve flap (5) is integrated in the balloon mouth (3).

7. Device according to claim 6, characterized in that the valve flap (5) integrated in the balloon mouth (3) is connected to the flexible elongated prolongation element (4) by force locking for the deflation of the balloon.

8. Device according to claim 1, characterized in that the balloon body (1) holds a volume between 0.25 and 9 ml, preferably between 1 and 3 ml, and/or the balloon mouth has an opening diameter between 1.0 and 6.0 mm, preferably between 1.5 and 3.0 mm, and/or the balloon body (1) comprises in its width and its length dimension a ratio between 7×10 mm and 70×120 mm.

9. Device according to claim 1, characterized in that the flexible elongated prolongation element (4) comprises a channel (6) for at least one section, which is in fluidic communication with the lumen of the balloon body (1) and at its distal end an auxiliary catheter can be introduced via an opening (7).

10. Device according to claim 9, characterized in that the channel (6) is formed for the entire length of the flexible elongated prolongation element (4) and a valve flap (5) is arranged at the distal end of the prolongation element (4) in the channel (6).

11. Device according to claim 9, characterized in that the channel (6) is formed only through the proximal part of the flexible elongated prolongation element (4) and not at the distal part of the flexible elongated prolongation element (4) and a valve flap (5) is arranged at the distal end of the channel (6).

12. Device according to claim 1, characterized in that the flexible elongated prolongation element (4) in its lengthwise dimension corresponds at least to a multiple of the length of the balloon body (1) and the flexible elongated prolongation element (4) consists either of solid material or comprises a channel (6) for at least a section on the inside.

13. (canceled)

14. The method according to claim 17, characterized in that the organ occlusion involves an intrauterine tracheal occlusion during the treatment of a congenital fetal diaphragmatic hernia.

15. The method according to claim 17, characterized in that the organ occlusion involves an occlusion of the upper respiratory passages for staunching of bleeding, an occlusion of the esophagus or an occlusion of the auditory canals.

16. The method according to claim 17, characterized in that the occlusion device is designed for use in prevention or treatment of an organ constriction or scar formation.

17. A method of occluding an organ or organ cavity of a mammal comprising the steps of:
positioning an occlusion device into a mammal in need of an organ occlusion at a location, said occlusion device having an inflation lumen sized and shaped for receiving an inflation fluid, and an identification or inflation fluid removal member extending from said inflation lumen; and
inflating said inflation lumen.

18. The method according to claim 17 wherein said identification or inflation fluid removal member is a flexible, elongated prolongation element.

19. The method according to claim 17 wherein said identification or inflation fluid removal member is positioned within said mammal to allow said mammal, or a person independent of said mammal, to apply a force thereto and cause said inflation lumen to deflate or be removed from said inflated position.

20. The method according to claim 17 further including the step of using said identification or inflation fluid removal member to locate said inflated lumen positioned within said mammal, using said identification or inflation fluid removal member to cause said inflated lumen to deflate, or combinations thereof.
